(19) United States
(12) Patent Application Publication
(10) Pub. No.: US 2012/0330023 A1
(43) Pub. Date: Dec. 27, 2012

(54) Alkyl Pyridinium Dicyanamides and Method For The Production Thereof

(75) Inventors: Christoph Täschler, Visp (CH); Gilbert Clausen, Glis (CH)

(73) Assignee: LONZA AG, Basel (CH)

(21) Appl. No.: 11/547,651

(22) PCT Filed: Apr. 6, 2005
(86) PCT No.: PCT/EP05/03620

§ 371 Date: Nov. 30, 2011

Related U.S. Application Data
(60) Provisional application No. 60/559,964, filed on Apr. 7, 2004.

(30) Foreign Application Priority Data
Apr. 6, 2004 (EP) 04008314.9

(51) Int. Cl. C07D 213/20

(52) U.S. Cl. 546/347

(57) ABSTRACT

Alkylpyridinium dicyanamide of the formula:

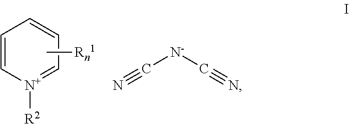

in which Rₙ¹ is cyano or C₁₋₂₀-alkyl and n is an integer from 0 to 3 and any R¹ radicals present are the same or different, and in which R² is C₁₋₂₀-alkyl. There are processes for their preparation from alkylpyridinium halides and alkali metal dicyanamide, They can be used as polar solvents.

The invention relates to alkylpyridinium dicyanamides of the formula

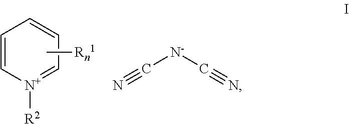

in which Rₙ¹ is cyano or C₁₋₂₀-alkyl and n is an integer from 0 to 3 and any R¹ radicals present are the same or different, and in which R² is C₁₋₂₀-alkyl. It further relates to their preparation from alkylpyridinium halides and alkali metal dicyanamides, and to their use as polar solvents.

Alkylpyridinium dicyanamides of the formula I are ionic liquids. Ionic liquids are commonly understood to mean salts which contain organic cations and are liquid at room temperature. The temperature range within which ionic liquids can be used as a solvent covers temperatures from −60° C. to over 300° C. As a result of their good solvation properties and their low volatility, they have become known in the last few years as environmentally friendly solvents for “green chemistry”.

Typically, the cations in ionic liquids are monovalent quaternary ammonium or phosphonium bases, or cations of aromatic nitrogen bases, which have optionally been substituted by alkyl groups, halogen atoms or cyano groups and may contain further heteroatoms such as O or S. Examples of nitrogen-containing cations are imidazolium, oxazolium, pyrazinium, pyrazolium, pyridazinium, pyridinium, pyrrolidinium, pyrimidinium, thiazolium and triazolium ions. Imidazolium and pyrrolidinium ions are the most frequently used. In structural formulae, the charge is shown localized on the heteroatom (usually on the nitrogen) or delocalized in the middle of the ring. The two illustrations are equivalent.

Typical anions in ionic liquids are acetate, AlCl₄⁻, AsF₆⁻, BF₄⁻, bromide, CF₃SO₃⁻, (CF₃)₂PF₄⁻, (CF₃)₃PF₃⁻(CF₃)₄PF₂⁻, (CF₃)₅PF⁻, (CF₃)₆P⁻, chloride, CN⁻, FeCl₃⁻, NO₃⁻, PF₆⁻, pyruvate, trifluoromethanesulphonate, oxalate or SCN⁻. The most frequently used are AlCl₄⁻, AsF₆⁻, BF₄⁻ and PF₆⁻.

Starting materials for the preparation of ionic liquids can be purchased, for example, from Merck KGaA, Darmstadt or IoLiTec, A. Bösmann, Dr T. Schubert G. b. R., Freiburg.

Ionic liquids with pyridinium ions and their use are disclosed, inter alia, in U.S. Pat. No. 2,455,331, WO-A1-02/34863, WO-A2-03/004727, US-A1-2004/0038031 and US-A1-2004/0031685.

Further known applications of ionic liquids based on pyridinium are disclosed in A. Ikeda et al. Chemistry Letters 2001, 1154-1155; M. Grätzel et al. J. Phys. Chem. B 107, 2003, 13280-13285; G. L. Rebeiro and B. M. Khadilkar Synthesis 3, 2001, 370-372; A. J. Carmichael and M. J. Earle Organic Letters 1, 1999, 997-1000; A. Boesmann et al. Angew. Chem. Int. Ed. 40, 2001, 2697-2699 and A. Eleuteri and D. Capaldi Org. Proc. Res. Dev. 4, 2000, 182-189.

Ionic liquids which contain a dicyanamide ion (N,N-dialkylimidazolium dicyanamide, N,N-dialkylpyrrolidinium dicyanamide and tetraalkylammonium dicyanamide) are disclosed in D. R. MacFarlane, et al. Chem. Commun. 2001, 1430-1431; S. A. Forsyth et al. Chem. Commun. 2002, 714-715 and D. R. MacFarlane, et al. Green Chemistry. 4, 2002, 444-448. All N,N-dialkylimidazolium dicyanamides, N,N-dialkylpyrrolidinium dicyanamides and tetraalkylammonium dicyanamides disclosed are prepared from the corresponding imidazolium, pyrrolidinium and tetraalkylammonium iodides with silver dicyanamide (Ag(C₂N₃)). Other preparation methods are not disclosed, nor are any examples for the preparation of the dicyanamides mentioned from other halides.

It is an object of the present invention to provide novel ionic liquids and inexpensive processes for their preparation. The new compounds should be disposable in an environmentally harmless manner after use.

This object is achieved in accordance with claim 1.

The compounds claimed are alkylpyridinium dicyanamides of the formula

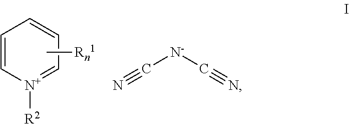

in which Rₙ¹ is cyano or C₁₋₂₀-alkyl and n is an integer from 0 to 3 and any R¹ radicals present are the same or different, and in which R² is C₁₋₂₀-alkyl.

Here and hereinafter, the expression “C₁₋ₙ-alkyl” means an unbranched or branched alkyl group having 1 to n carbon atoms. C₁₋₂₀-alkyl represents, for example, methyl, ethyl, propyl, isopropyl, butyl, isobutyl, sec-butyl, tert-butyl, pentyl, 1,4-dimethylpentyl, hexyl, heptyl, octyl, 1,5-dimethylhexyl, nonyl, decyl and 4-ethyl-1,5-dimethylhexyl, undecyl, dodecyl, tridecyl, tetradecyl or eicosyl.

Preferred alkylpyridinium dicyanamides are compounds of the formula I where Rₙ¹ is cyano or C₁₋₈-alkyl, preferably methyl or ethyl, and n is an integer from 0 to 2 and any R¹ radicals present are the same or different, and in which R² is C₂₋₈-alkyl. Particular preference is given to alkylpyridinium dicyanamides according to the formula

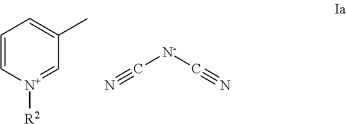

in which R² is C₄₋₈-alkyl.

The anions used customarily in ionic liquids have the disadvantage that their thermal disposal often gives rise to halogenated and metallic residues. When the inventive alkylpyridinium dicyanamides are disposed of thermally, these wastes do not arise.

By virtue of the aromatic structure, the inventive ionic liquids having optionally substituted pyridine-based cations open up a wider polarity range than, for example, those formed from quaternary ammonium ions.

The water solubility of the inventive alkylpyridinium dicyanamides can be adjusted by virtue of the number and chain length of the substituents Rₙ¹ and R² in the inventive compounds within a range from “completely water-miscible” up to “water-immiscible”. For example, N-butylpyridinium dicyanamide and N-butyl-3-methylpyridinium dicyanamide are water-miscible, while N-octyl-3-methylpyridinium dicyanamide and N-octylpyridinium dicyanamide are water-immiscible. The miscibility with solvents, for example acetone, acetonitrile, DMSO, ethyl acetate, hexane, methylene chloride, organic acids, propylene carbonate, carbon disulphide, THF, toluene or other ionic liquids is likewise determined significantly by the side groups Rₙ¹ and R².

A process for preparing the inventive alkylpyridinium dicyanamides of the formula

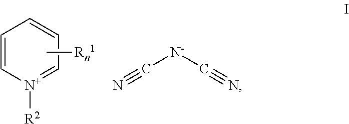

in which Rₙ¹ is cyano or C₁₋₁₀-alkyl and n is an integer from 0 to 3 and any R¹ radicals present are the same or different, and in which R² is C₁₋₁₀-alkyl, comprises the reaction of a compound of the formula

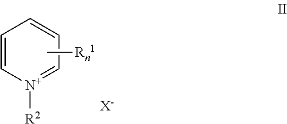

where Rₙ¹ and R² are each as defined above and X is a halogen atom selected from the group consisting of fluorine, chlorine, bromine and iodine, with an alkali metal dicyanamide, preferably in the presence of water.

Here and hereinafter, alkali metal dicyanamides are understood to mean dicyanamides of the alkali metals lithium, sodium, potassium, and the mixtures and hydrates thereof. The alkali metal dicyanamides may be used as a solid, in solution or suspension; they are more preferably used as an aqueous solution or suspension.

In a preferred embodiment, the molar ratio of the reactants alkylpyridinium halide: alkali metal dicyanamide is within a range of 0.2:1 to 5:1, more preferably in the ratio of 1:1. Unconverted starting materials can be removed in a simple manner from the alkylpyridinium dicyanamide formed.

In a preferred process variant, the reaction of alkylpyridinium halide and alkali metal dicyanamide is performed in the presence of water in a molar ratio of the sum of the reactants to water of 2:0 to 2:100, preferably of 2:10 to 2:30, more preferably of 2:15 to 2:25.

Compared to the known process for preparing ionic liquids with dicyanamide ions, the process according to the invention is notable in that the preparation of Ag(C₂N₃) as an intermediate compound can be dispensed with.

As a result of the high concentration of the starting compounds in the process according to the invention, alkylpyridinium dicyanamides can be prepared in a surprisingly simple and inexpensive manner directly from alkylpyridinium halides and alkali metal dicyanamides. The expensive workup of the silver-containing wastes which occur is thus also dispensed with. Moreover, it is possible in the process according to the invention to use not just iodides but also the far more favourable other halides without yield losses. In a further preferred process variant, depending on the number and properties of the Rₙ¹ and R² radicals, in addition to water, further solvents, for example acetone, acetonitrile, C₁₋₄-alcohols, chloroform, dichloroethane, diethyl ether, DMSO, ethyl acetate, hexane, methylene chloride, propylene carbonate, carbon disulphide, THF, toluene and/or xylene are also used as solubilizers.

In a preferred embodiment, the alkylpyridinium dicyanamides are purified by an extraction process. More preferably, alkylpyridinium dicyanamides and alkali metal halides which form can be isolated in the presence of water by phase separation. When water is already present in the reaction solution, even alkylpyridinium dicyanamides which are otherwise entirely water-miscible, as a result of alkali metal halide formed during the reaction, form a phase interface (salting-out effect). This salting-out effect can be enhanced by salt addition, for example during the extraction.

Moreover, it has been found that, surprisingly, even readily water-soluble alkylpyridinium dicyanamides can be extracted from aqueous solutions with water-immiscible organic solvents of low polarity, for example methylene chloride.

Pyridine and various alkylpyridines occur in natural sources, for example in coal tar, and are isolated there in a technically simple manner and in large amounts. Since imidazoles are not naturally occurring, the inventive alkylpyridinium dicyanamides have a cost advantage over known imidazolium dicyanamides. Substituted pyridines can be prepared by a multitude of methods. The N-alkylation of the pyridines is likewise effected by known processes, for example with alkyl halides. Since pyridines have only one alkylatable nitrogen atom in the ring, homogeneous products are obtained specifically in the N-alkylation, while the N-alkylation of substituted imidazole derivatives can often lead to inhomogeneous products which are difficult to separate.

The inventive alkylpyridinium dicyanamides of the formula

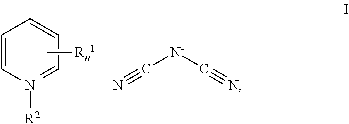

in which Rₙ¹ is cyano or C₁₋₁₀-alkyl and n is an integer from 0 to 3 and any R¹ radicals present are the same or different, and in which R² is C₁₋₁₀-alkyl, are ionic liquids and can be used if appropriate in a mixture with one or more other ionic liquids, water or organic solvents. Possible uses of the inventive alkylpyridinium dicyanamides are, for example, as a constituent of polar solvents, as electrolytes in electrolysis or in electrical components or for the production of liquid crystals for LCDs or of conductive gels, for example for applications in photovoltaics.

It has also been found that, in Suzuki reactions (C—C bond formation of haloaromatics with aromatic boric esters), a smaller amount of by-products are generated in the inventive ionic liquids than when ionic liquids based on the known imidazolium dicyanamides are used.

The examples which follow are intended to illustrate the invention but without constituting a restriction.

### EXAMPLES

### Example 1

### 3-Methyl-1-butylpyridinium chloride (Formula II, R¹=3-methyl, R²=butyl, X=Cl)

3-Methylpyridine (2142 g, 23 mol) and 1-chlorobutane (2129 g, 23 mol) are initially charged in a 5 l jacketed stirred apparatus and stirred under reflux. The temperature in the stirred apparatus is approx. 92-95° C. The initially clear solution forms initially 2 phases, of which the lower comprises the product. After full conversion, only one phase is present. The product can be separated from the starting materials in a simple manner batchwise or continuously by phase separation. After 33 h, 1042 g of crude 3-methyl-1-butylpyridinium chloride are obtained. Residues of 3-methylpyridine and 1-chlorobutane (<5%) are removed by vacuum treatment at approx. 90° C. The purity by ¹H NMR is >98%.

### Example 2

### 3-Methyl-1-butylpyridinium dicyanamide (Formula I, R¹=3-methyl, R²=butyl)

An aqueous solution of 3-methyl-1-butylpyridinium chloride (1686 g, 9 mol) in water (2550 ml, 140 mol) is admixed at room temperature in portions with solid sodium dicyanamide (808 g, 9 mol) and then stirred until all solids dissolve. This forms a clear beige solution. The solution is admixed with 85 g of activated carbon and stirred for 30 min and then filtered. The clear yellowish solution is then extracted with dichloromethane each time (2×1000 ml). The extracts are then washed with water (3×500 ml) and then concentrated to constant weight first at 50° C. and 500 mbar, later at 20 mbar. In this way, 1708 g of 3-methyl-1-butylpyridinium dicyanamide are obtained in the form of a light beige liquid, corresponding to 87% yield and with a purity of >98% by ¹H NMR.

### Example 3

### 3-Methyl-1-octylpyridinium chloride (Formula II, R¹=3-methyl, R²=octyl, X=Cl)

3-Methylpyridine (1676 g, 18 mol) and 1-chlorooctane (2676 g, 18 mol) are initially charged in a 5 l jacketed stirred apparatus and stirred at a temperature of 130-135° C. The initially clear solution forms 2 phases, of which the lower contains the 3-methyl-1-octyl-pyridinium chloride. In the course of the reaction, the upper phase decreases ever further until the reaction mixture in turn consists only of one phase after full conversion. After 24 h, 4370 g of crude product are obtained. The resulting beige to brownish reaction product solidifies gradually in the course of cooling to room temperature. Purity by ¹H NMR>98%, melting range 63-70° C.

### Example 4

### 3-Methyl-1-octylpyridinium dicyanamide (Formula I, R¹=3-methyl, R²=octyl)

A mixture of 3-methyl-1-octylpyridinium chloride (242 g, 1 mol) and a little water (25 ml, 1.4 mol) is admixed with a saturated solution of sodium dicyanamide (89 g, 1 mol) in water (340 ml, 18.9 mol) at room temperature within 30 min. After completion of metered addition, the reaction mixture is stirred for a further 20 min. This forms two phases. The (lower) organic phase is removed and the aqueous phase is extracted with dichloromethane (2×300 ml). The dichloromethane extracts and the organic phase removed beforehand are combined and washed with water (3×250 ml). The organic phase is concentrated to constant weight initially at 500 mbar and 50° C., later at 20 mbar and 50° C. In this way, 218 g of a light beige clear liquid (corresponding to 90% yield) of 3-methyl-1-octylpyridinium dicyanamide are obtained with a purity of >98% by NMR.

1. Alkylpyridinium dicyanamide of the formula: 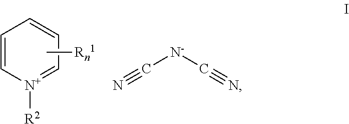
in which Rₙ¹ is cyano or C₁₋₂₀-alkyl and n is an integer from 0 to 3 and any R¹ radicals present are the same or different, and in which R² is C₁₋₂₀-alkyl.

2. The alkylpyridinium dicyanamide according to claim 1, in which Rₙ¹ is cyano or C₁₋₈-alkyl, and n is an integer from 0 to 2 and any R¹ radicals present are the same or different, and in which R² is C₂₋₈-alkyl.

3. Alkylpyridinium dicyanamide of the formula: 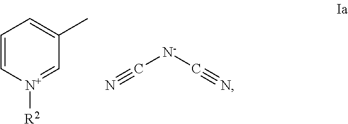
wherein R² is C₄₋₈-alkyl.

4. Process for preparing the alkylpyridinium dicyanamide according to claim 1, comprising reacting an alkylpyridinium halide of the formula: 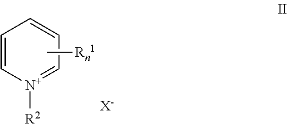
wherein Rₙ¹ is cyano or C1 20 alkyl and n is an integer from 0 to 3 and any R¹ radicals present are the same or different, and in which R² is C₁₋₂₀-alkyl, and in which X⁻ is a halogen ion, with an alkali metal dicyanamide.

5. The process according to claim 4, wherein X⁻ is chloride or bromide.

6. The process according to claim 5, wherein the molar ratio of alkylpyridinium halide: alkali metal dicyanamide is within a range of 0.2:1 to 5:1.

7. The process according to claim 6, wherein the reaction of alkylpyridinium halide and alkali metal dicyanamide is performed in the presence of water in a molar ratio of the sum of the reactants to water of 2:0 to 2:100.

8. The process according to claim 7, wherein the alkali metal dicyanamide is used in the form of a solution or suspension.

9. Process comprising utilizing the alkylpyridinium dicyanamide according to claim 1, if appropriate in a mixture with one or more other ionic liquids, water or organic solvents, as a polar aprotic solvent, as an electrolyte in electrolysis or in electrical components, or for the production of liquid crystals for LCDs or of conductive gels.

10. Process comprising utilizing the alkylpyridinium dicyanamide according to claim 1, if appropriate in a mixture with one or more other ionic liquids, water or organic solvents, as a solvent in a Suzuki reaction.

11. The alkylpyridinium dicyanamide according to claim 2, wherein Rₙ¹ is methyl or ethyl.

12. The process according to claim 4, wherein the reaction is conducted in the presence of water.

13. The process according to claim 5, wherein the molar ratio of alkylpyridinium halide: alkali metal dicyanamide is in the ratio of 1:1.

14. The process according to claim 4, wherein the molar ratio of alkylpyridinium halide: alkali metal dicyanamide is within a range of 0.2:1 to 5:1.

15. The process according to claim 14, wherein the molar ratio of alkylpyridinium halide: alkali metal dicyanamide is the ratio of 1:1.

16. The process according to claim 6, wherein the reaction of alkylpyridinium halide and alkali metal dicyanamide is performed in the presence of water in a molar ratio of the sum of the reactants to water of 2:10 to 2:30.

17. The process according to claim 16, wherein the alkali metal dicyanamide is used in the form of a solution or suspension.

18. The process according to claim 4, wherein the reaction of alkylpyridinium halide and alkali metal dicyanamide is performed in the presence of water in a molar ratio of the sum of the reactants to water of 2:15 to 2:25.

19. The process according to claim 18, wherein the alkali metal dicyanamide is used in the form of a solution or suspension.

20. The process according to claim 4, wherein the molar ratio of alkylpyridinium halide: alkali metal dicyanamide is within a range of 0.2:1 to 5:1.

21. The process according to claim 20, wherein the molar ratio of alkylpyridinium halide: alkali metal dicyanamide is the ratio of 1:1.

22. The process according to claim 4, wherein the reaction of alkylpyridinium halide and alkali metal dicyanamide is performed in the presence of water in a molar ratio of the sum of the reactants to water of 2:10 to 2:30.

23. The process according to claim 22, wherein the alkali metal dicyanamide is used in the form of a solution or suspension.

24. The process according to claim 4, wherein the reaction of alkylpyridinium halide and alkali metal dicyanamide is performed in the presence of water in a molar ratio of the sum of the reactants to water of 2:15 to 2:25.

25. The process according to claim 24, wherein the alkali metal dicyanamide is used in the form of a solution or suspension.

26. The process according to claim 4, wherein the alkali metal dicyanamide is used in the form of a solution or suspension.
